SEQUENCE LISTING (2 sequences)

SEQ ID NO: 1 - protein, 12 aa, Artificial Sequence
  Synthetic
LMKNMDPLNDNV

SEQ ID NO: 2 - DNA, 36 bp, Artificial Sequence
  Synthetic DNA
ytnatgaaraayatggayccnytnaaygayaaygtn

(19) United States
(12) Patent Application Publication
(10) Pub. No.: US 2015/0284453 A1
(43) Pub. Date: Oct. 8, 2015

(54) METHOD OF PROTECTING CARDIAC FUNCTION

(71) Applicant: Declmmune Therapeutics, Inc., Cambridge, MA (US)

(72) Inventor: Michael S. Haas, Chelmsford, MA (US)

(21) Appl. No.: 14/670,700

(22) Filed: Mar. 27, 2015

Related U.S. Application Data
(63) Continuation of application No. PCT/US2013/062553, filed on Sep. 30, 2013.
(60) Provisional application No. 61/708,403, filed on Oct. 1, 2012.

(51) Int. Cl. C07K 16/18

(52) U.S. Cl. CPC C07K 16/18; C07K 2317/565; C07K 2317/20; A61K 2039/505

(57) ABSTRACT

The invention is a method of protecting cardiac function in a subject in need thereof comprising administering to said subject an effective amount of an inhibitor of the interaction between a pathogenic IgM and the N2 epitope in cardiac tissue.

### RELATED APPLICATION

This application is a continuation of International Application No. PCT/US2013/062553, which designated the United States and was filed on Sep. 30, 2013, published in English, which claims the benefit of U.S. Provisional Application No. 61/708,403, filed on Oct. 1, 2012. The entire teachings of the above applications are incorporated herein by reference.

### GOVERNMENT SUPPORT

The invention was supported, in whole or in part, by Grant No. 5R44HL084821-03 from the National Institutes of Health. The Government has certain rights in the invention.

### BACKGROUND OF THE INVENTION

Reperfusion therapies, such as primary percutaneous coronary intervention and thrombolytic therapy, are a mainstay in the treatment of patients who have suffered myocardial infarction. However, myocardial reperfusion is itself associated with injury (Hausenloy et al. (2008), NJEM, 359(5): 518-520 and Yellon et al. (2007), NJEM 357(11): 1121-1135). This reperfusion injury can have a significant effect on clinical outcome as the coincident injury that occurs during revascularization can result in further impairment of cardiac function (Id.). There is therefore a need in the art for therapies focused on mitigating injury due to revascularization.

### SUMMARY OF THE INVENTION

The present invention is based on the discovery that administration of an isolated N2 peptide and the murine 21G6 antibody (inhibitors of the interaction between N2 epitope and pathogenic IgM) when administered prior to reperfusion results in significant protection in left ventricular ejection fraction (LVEF), a measure of cardiac function. Specifically, as described in Example 1, in a porcine model of myocardial infarction, N2- and murine 21G6-treated animals recovered over 98% of baseline LVEF as compared to about 75% in the saline-treated animals.

Accordingly, in one embodiment, the invention is a method of protecting ejection fraction in a subject that has suffered myocardial infarction comprising administering to said subject an effective amount of an inhibitor of the interaction between a pathogenic IgM and the N2 epitope in cardiac tissue prior to and/or during reperfusion therapy. In some embodiments, the ejection fraction is LVEF. In certain embodiments, the inhibitor is administered prior to reperfusion. In yet additional embodiments, the inhibitor is administered during reperfusion.

In another aspect, the invention is a method of protecting cardiac function in a subject in need thereof comprising administering to said subject an effective amount of an inhibitor of the interaction between a pathogenic IgM and the N2 epitope in cardiac tissue. In one embodiment, the protection of cardiac function comprises a protection from loss of ejection fraction (for example, LVEF) and/or fractional shortening. In some embodiments, the patient is suffering from, or at risk of suffering from an ischemic cardiovascular event. In other embodiments, the patient is at risk for reperfusion injury. In certain embodiments, the inhibitor is administered prior to and/or during reperfusion. In another aspect, the inhibitor is administered before and/or during a surgical procedure.

In a further aspect, the invention is a method of treating a subject at risk for reduced cardiac function after reperfusion comprising administering to said patient an effective amount of an inhibitor of the interaction between a pathogenic IgM and the N2 epitope in cardiac tissue prior to and/or during reperfusion therapy. In some aspects, the patient is at risk for reduced LVEF. In yet other aspects, the inhibitor is administered prior to reperfusion.

In certain aspects, the inhibitor is a peptide inhibitor, for example, an isolated peptide comprising SEQ ID NO: 1, wherein the peptide is less than 50 amino acids in length. In additional aspects, the inhibitor is an isolated antibody or an antigenic fragment thereof. In one embodiment, the antibody or fragment thereof possesses the antigenic specificity of the 12A6 antibody or the 21G6 antibody. In yet another embodiment, the antibody or fragment thereof possesses the epitopic specificity of the 12A6 antibody or the 21G6 antibody. In certain aspects, the antibody or fragment thereof comprises heavy chain CDR1, CDR2 and CDR3 each having the same amino acid sequence as the heavy chain CDR1, CDR2, and CDR3, respectively, of 21G6 and comprises light chain CDR1, CDR2 and CDR3 each having the same amino acid sequence as the light chain CDR1, CDR2, and CDR3, respectively, of 21G6, respectively. In certain aspects, the antibody or fragment thereof comprises heavy chain CDR1, CDR2 and CDR3 having the same amino acid sequence as the heavy chain CDR1, CDR2, and CDR3, respectively, of 12A6 and comprising light chain CDR1, CDR2 and CDR3 having the same amino acid sequence as the light chain CDR1, CDR2, and CDR3, respectively, of the 12A6 antibody.

### BRIEF DESCRIPTION OF THE DRAWINGS

FIG. 1 shows images of tissue sections of pigs subjected to 1 h left anterior descending (LAD) occlusion and 1 h reperfusion. Prior to reperfusion, fluorescently labeled murine 21G6 mAb (arrow heads, red) was injected intravenously (i.v.). Tissue sections were also stained with CD31 (arrows, green) to visualize vasculature. “LV” designates left ventricle; “RV” designates right ventricle. Scale bar, 20 μm.

FIG. 2A to 2C are plots showing that N2 peptide and murine 21G6 mAb reduce infarct size in a swine model of myocardial infarction. Swine were subjected to 1 h LAD occlusion and 5 d reperfusion. Prior to reperfusion either saline, N2 (4.6 mg/kg), or murine 21G6 mAb (2 mg/kg) was injected i.v. into the swine. Following 5 d reperfusion, myocardial sections were stained with Evan's blue and TTC. (A) Myocardial infarct size expressed as a percentage of the left ventricle (LV). (B) Percentage of area at risk (AAR) to the LV. (C) Myocardial infarct size expressed as a percentage of the AAR. # represents p<0.05; ## represents p<0.01; ### represents p<0.001. Each symbol represents data from one animal. (Note: Due to poor perfusion with Evan's blue dye in one animal, n=5 in panels B and C).

FIGS. 3A and 3B are plots showing the effect of N2 peptide and murine 21G6 mAb (m21G6) on serum cardiac troponin-T (cTnT) levels. Swine were subjected to 1 h LAD occlusion and 5 d reperfusion. Prior to reperfusion either saline, N2 (4.6 mg/kg), or m21G6 (2 mg/kg) was injected i.v. into the swine. Serum samples were taken at several time points as described in text and analyzed for cTnT. (A) Peak cTnT levels. (B) cTnT levels over 5d reperfusion. # represents p<0.05; ## represents p<0.01. Each symbol represents data from one animal.

FIG. 4A to 4C are graphs showing measurements of cardiac function by echocardiography. (A) Non-corrected % LVEF as measured by 2D TTE at 21d post-reperfusion compared to baseline using Quinones method (described below in the Examples section). (B) % LVEF as measured by 3D TTE at 21d post-reperfusion. (C) Fractional shortening as measured by 2D TTE at 21d post-reperfusion compared to baseline. # represents a p<0.05. n=3/group.

### DETAILED DESCRIPTION OF THE INVENTION

The words “a” or “an” are meant to encompass one or more, unless otherwise specified.

An “isolated” molecule, e.g., an isolated antibody or isolated peptide, refers to a condition of being separate or purified from other molecules present in the natural environment or as they occur in nature.

Myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heart beat. The term ejection fraction applies to both the right and left ventricles. LVEF refers to the left ventricular ejection fraction (LVEF). Fractional shortening refers to the difference between end-diastolic and end-systolic dimensions divided by end-diastolic dimension.

Protecting cardiac function refers to reducing or preventing the deterioration in cardiac function that normally accompanies an event, such as an ischemic cardiac event and/or reperfusion. Protecting cardiac function can comprise protecting at least one of ejection fraction (for example, LVEF) or fractional shortening from loss, or in other words, reducing or preventing an impairment (decrease) in ejection fraction or fractional shortening. Cardiac function measurements (for example, LVEF) are taken at least three weeks after the ischemic event and/or after reperfusion. LVEF can be measured, for example, by echocardiography. A normal LVEF in humans is about 55 to about 70%. As described above, reperfusion injury and/or ischemic cardiac events are associated with a decrease in cardiac function, such as a decrease in LVEF. As used herein, LVEF is protected from loss according to a method of the invention when the LVEF is higher than it would have been in the absence of inhibitor administration (when measured at least three weeks after the ischemic cardiac event and/or reperfusion). LVEF is protected from loss when the loss of LVEF that would have occurred, for example, due to revascularization, is reduced. It is to be understood that LVEF is also protected from loss when there is no loss in LVEF after reperfusion as compared with that before reperfusion. Protecting LVEF, or protecting LVEF from loss after reperfusion, can encompass maintaining an LVEF within about 15% or less, 10% or less, 5%, or less, or 0% of that before reperfusion. In another example, protecting LVEF or reducing an impairment in LVEF, after reperfusion encompasses retaining about 80% or more, about 85% or more, about 90% or more, about 95% or more, or about 97% or more of the LVEF before reperfusion.

The IgM inhibitor can be administered prior to the time of tissue injury or when tissue injury (for example, reperfusion and/or a surgical procedure) is expected to occur. The IgM inhibitor can also, or alternatively, be administered contemporaneously with tissue injury or when tissue injury is expected to occur, for example, during a surgical procedure or reperfusion. In specific examples, the inhibitor is administered prior to and/or during reperfusion. For example, in a patient at risk for reperfusion injury (for example, a patient being prepared by his/her physician for reperfusion therapy), the inhibitor is administered prior to, or during reperfusion. Alternatively, the inhibitor can be administered prior to and during reperfusion. The inhibitor can, for example, be administered intravenously. The person of skill in the art will appreciate that the amount of time before reperfusion or expected tissue injury at which the inhibitor is administered will depend on the half-life (t_{1/2}) of the inhibitor. A peptide inhibitor could, for example, be administered within minutes of the initiation of reperfusion, for example five minutes before the initiation of reperfusion. An antibody inhibitor could, for example, be administered within a few hours of the initiation of reperfusion.

In some embodiments, the subject is a human subject. In certain additional embodiments, the subject has suffered myocardial infarction and the inhibitor is administered after the subject has suffered myocardial infarction. In further embodiments, the subject has suffered myocardial infarction and the inhibitor is administered before reperfusion therapy. In an additional embodiment, the subject is suffering from or at risk of suffering from an ischemic cardiac event such as valve replacement and coronary artery bypass graft (CABG). In yet another aspect, the subject is to undergo any surgical procedure that disrupts blood flow to the heart. In some aspects, the subject is at risk for reperfusion injury. A subject is at risk for reperfusion injury or an ischemic cardiac event, for example, when the subject is to undergo reperfusion therapy or a surgical procedure (such as CABG or valve replacement) in the future.

As described above, the present invention is directed to the administration of an inhibitor of the interaction between a pathogenic IgM and the N2 epitope on non-muscle myosin heavy chain (NMHC) type II in cardiac tissue. N2 is a self-antigen, an antigen expressed or exposed on damaged ischemic tissue, for example on damaged cardiac tissue. The N2 epitope is an epitope of the self-antigen, the 12 amino acid sequence expressed in NMHC type II. The 12-amino acid sequence is LMKNMDPLNDNV (SEQ ID NO: 1). Pathogenic IgM (also referred to herein as “natural IgM”) recognizes and binds N2 expressed or exposed on damaged tissue, and in particular damaged ischemic tissue, and thereby initiates inflammation by activating complement in the classical pathway. The inhibitors that can be used according to the present invention can, for example, compete with pathogenic IgM antibodies in binding the N2 epitope, thereby titrating out N2 antigen available to bind IgM and activate complement. The inhibitors can also, for example, bind pathogenic IgM antibodies and thereby reduce the amount of pathogenic IgM available to bind to self-antigen.

“Natural IgM” or “pathogenic IgM” as used herein to refer to an IgM antibody that is naturally produced in a mammal (e.g., a human) that binds to the N2 epitope and initiates inflammation by activating complement in the classical pathway. Production of natural IgM antibodies in a subject is important in the initial activation of B-cells, macrophages, and the complement system.

The inhibitor can also be referred to herein as an “IgM inhibitor” or “inhibitor.” Inhibitors have been described, for example, U.S. Pat. No. 7,442,783, U.S. Patent Application Publication No. 20090176966 and U.S. Patent Application Publication No. 20120093835A1, the contents of each of which are expressly incorporated by reference herein. The inhibitor can, for example, be a protein or a peptide, an antibody or fragment thereof, a modified antibody, a carbohydrate, a glycoprotein, or a small organic molecule.

In one embodiment, the IgM inhibitor is a peptide that specifically binds to a natural IgM and thereby blocks binding to the N2 antigen and/or complement activation and/or ischemic injury. An example of a peptide that can be used according to the present invention is an isolated peptide comprising the amino acid sequence of SEQ ID NO: 1, wherein the peptide is less than about 50 amino acids in length. In some embodiments, the peptide is less than about 45, 40, 35, 30, 25, 20, or 15 amino acids in length. In another embodiment, the peptide is s peptide consisting of an amino acid sequence having SEQ ID NO: 1. In yet another embodiment, the peptide comprises an amino acid sequence having at least about 80%, 85%, 90%, 95%, or 98% sequence identity to the amino acid sequence of SEQ ID NO: 1, wherein the peptide is less than about 50 amino acids in length. In a further embodiment, the peptide consists of an amino acid sequence having at least about 80%, 85%, 90%, 95%, or 98% sequence identity to the amino acid sequence of SEQ ID NO: 1. In some embodiments, the peptide is less than about 45, 40, 35, 30, 25, 20, or 15 amino acids in length.

When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md.

The inhibitor of the interaction of the N2 epitope and a pathogenic IgM can also be an antibody inhibitor. An antibody is a binding molecule and includes immunoglobulin molecules, antibody fragments, and immunologically active portions of immunoglobulin molecules, i.e., molecules that contain an antigen-binding site. Antibodies useful in the invention can be of any class (for example, IgG, IgE, IgM, IgD, and IgA) or subclass. In some embodiments, the antibody is an IgG. Native antibodies and immunoglobulins are usually heterotetrameric glycoproteins of about 150,000 Daltons, composed of two identical light chains and two identical heavy chains. Each heavy chain has at one end a variable domain followed by a number of constant domains. Each light chain has a variable domain at one end and a constant domain at its other end. Antibodies include, but are not limited to, polyclonal, monoclonal, bispecific, chimeric, partially or fully humanized antibodies, fully human antibodies (i.e., generated in a transgenic mouse expressing human immunoglobulin genes), camel antibodies, and anti-idiotypic antibodies. An antibody, or generally any molecule, “binds specifically” to an antigen (or other molecule) if the antibody binds preferentially to the antigen, and, e.g., has less than about 30%, preferably 20%, 10%, or 1% cross-reactivity with another molecule. The terms “antibody” and “immunoglobulin” are used interchangeably.

“Antibody fragment” is used herein to refer to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)₂, scFv, Fv, dsFv diabody, minibody, Fc, Fd fragments, and single chain antibodies. The antibody fragment can be produced by any means. For instance, the antibody fragment can be enzymatically or chemically produced by fragmentation of an intact antibody, it can be recombinantly produced from a gene encoding the partial antibody sequence, or it may be wholly or partially synthetically produced. The antibody fragment can optionally be a single chain antibody fragment. Alternatively, the fragment can comprise multiple chains which are linked together, for instance, by disulfide linkages. The fragment can also optionally be a multimolecular complex. A functional antibody fragment will typically comprise at least about 50 amino acids and more typically will comprise at least about 200 amino acids.

An antibody inhibitor can be an isolated antibody or antigen-binding fragment thereof that binds specifically to amino acid sequence of SEQ ID NO: 1. In another aspect, the antibody specifically binds to an amino acid sequence encoded by a nucleic acid comprising YTN ATG AAR AAY ATG GAY CCN YTN AAY GAY AAY GTN (SEQ ID NO: 2), where an “R” corresponds to a base that may be an A or G; a “Y” corresponds to a base that may be a C or T; and an “N” corresponds to a base that may be an A, C, G or T, and is capable of inhibiting inflammation in a subject to whom the antibody is administered. Non-limiting examples of antibody inhibitors are described, for example, in U.S. Pat. No. 7,442,783, U.S. Patent Application Publication Nos. 20090176966A1 and 20120093835-A1. As described in U.S. Patent Application Publication No. 20120093835A1, anti-N2 antibodies can be obtained from a hybridoma that has been deposited with the American Type Culture Collection and provided Accession Number PTA-9392 (IgG^{12A6} or 12A6) or PTA-9393 (IgG^{21G6} or 21G6). The nucleic acid and amino acid sequences of these antibodies, including the complementarity determining regions (CDRs) are also described in U.S. Patent Application Publication No. 20120093835A1.

In some embodiments, the antibody inhibitor comprises a complementarity determining region (CDR1, CDR2, CDR3 of the light chain or heavy chain) that has the same amino acid sequence as the CDR1, CDR2, or CDR3 of the light chain or heavy chain of the 12A6 or 21G6 antibodies. In certain embodiments, the antibody inhibitor is a human or humanized antibody.

As described in U.S. Patent Application Publication No. 20120093835A1, the nucleic acid encoding the heavy or light chain variable region can be of murine or human origin, or can comprise a combination of murine and human amino acid sequences. For example, the nucleic acid can encode a heavy chain variable region comprising the CDR1, CDR2, and/or CDR3 of the 21G6 antibody and a human framework sequence. In addition, the nucleic acid can encode a light chain variable region comprising the CDR1, CDR2 and/or CDR3 of the 21G6 antibody and a human framework sequence. The nucleic acid encoding the heavy or light chain variable region can be of murine or human origin, or can comprise a combination of murine and human amino acid sequences. For example, the nucleic acid can encode a heavy chain variable region comprising the CDR1, CDR2, and/or CDR3 of the 12A6 antibody and a human framework sequence. In addition, the nucleic acid can encode a light chain variable region comprising the CDR1, CDR2 and/or CDR3 of the 12A6 antibody and a human framework sequence. The invention further encompasses vectors containing the above-described nucleic acids and host cells containing the expression vectors.

In some embodiments of the present invention, antibody inhibitors that immunospecifically bind to the N2 self-peptide (SEQ ID NO: 1) comprise a VH CDR1 that has the same amino acid sequence as the VH CDR1 of 21G6 antibody. In another embodiment, antibodies that immunospecifically bind to the N2 self-peptide comprise a VH CDR2 that has the same amino acid sequence as the VH CDR2 of the 21G6 antibody. In another embodiment, antibodies that immunospecifically bind to the N2 self-peptide comprise a VH CDR3 that has the same amino acid sequence of the VHCDR1 of the 21G6 antibody.

In one embodiment of the present invention, antibodies that immunospecifically bind to an N2 self-peptide (SEQ ID NO: 1) comprise a VL CDR1 that has the same amino acid sequence as the VL CDR1 of the 21G6 antibody. In another embodiment, antibodies that immunospecifically bind to the N2 self-peptide comprise a VL CDR2 that has the same amino acid sequence as the VLCDR2 of the 21G6 antibody. In another embodiment, antibodies that immunospecifically bind to the N2 self-peptide comprise a VL CDR3 that has the same amino acid sequence of the VL CDR3 of the 21G6 antibody.

In some embodiments of the present invention, antibody inhibitors that immunospecifically bind to the N2 self-peptide comprise a VH CDR1 that has the same amino acid sequence as the VH CDR1 of the 12A6 antibody. In another embodiment, antibodies that immunospecifically bind to the N2 self-peptide comprise a VH CDR2 that has the same amino acid sequence as the VH CDR2 of the 12A6 antibody. In another embodiment, antibodies that immunospecifically bind to the N2 self-peptide comprise a VH CDR3 that has the same amino acid sequence as the VH CDR1 of the 12A6 antibody.

In one embodiment of the present invention, antibodies that immunospecifically bind to an N2 self-peptide comprise a VL CDR1 that has the same amino acid sequence as the VL CDR1 of the 12A6 antibody. In another embodiment, antibodies that immunospecifically bind to the N2 self-peptide comprise a VL CDR2 that has the same amino acid sequence as the VL CDR2 of the 12A6 antibody. In another embodiment, antibodies that immunospecifically bind to the N2 self-peptide comprise a VL CDR3 that has the same amino acid sequence as the VL CDR3 of the 12A6 antibody.

In one embodiment, the antibody inhibitor comprises a VH CDR1 of the 21G6 antibody and a VL CDR1, VL CDR2 and/or VL CDR2 of the 21G6 antibody. In another embodiment, an antibody inhibitor comprises a VH CDR2of the 21G6 antibody and a VL CDR1, VL CDR2 and/or VL CDR2 of the 21G6 antibody. In yet another embodiment, an antibody of the present invention comprises a VH CDR3 of the 21G6 antibody and a VL CDR1, VL CDR2 and/or VL CDR2 of the 21G6 antibody.

In another embodiment, the antibody inhibitor comprises a VH CDR1 of the 12A6 antibody and a VL CDR1, VL CDR2 and/or VL CDR2 of the 12A6 antibody. In another embodiment, an antibody inhibitor comprises a VH CDR2 of the 12A6 antibody and a VL CDR1, VL CDR2 and/or VL CDR2 of the 12A6 antibody. In yet another embodiment, an antibody of the present invention comprises a VH CDR3 of the 21G6 antibody and a VL CDR1, VL CDR2 and/or VL CDR2 of the 12A6 antibody.

In an additional embodiment, the antibody or fragment thereof has heavy chain CDR1, CDR2, and CDR3 that have the same amino acid sequence of the heavy chain CDR1, CDR2 and CDR3 of the 21G6 antibody and has the light chain CDR1, CDR2, and CDR3 that have the same amino acid sequence of the light chain CDR1, CDR2 and CDR3 of the 21G6 antibody. In yet an additional embodiment, the antibody or fragment thereof has heavy chain CDR1, CDR2, and CDR3 that have the same amino acid sequence of the heavy chain CDR1, CDR2 and CDR3 of the 12A6 antibody and has the light chain CDR1, CDR2, and CDR3 that have the same amino acid sequence of the light chain CDR1, CDR2 and CDR3 of the 12A6 antibody.

In another embodiment, the antibody inhibitor possess the same antigenic or epitopic specificity as the 12A6 antibody or 21G6 antibody.

Methods of producing antibodies are well known in the art. For example, a monoclonal antibody against a target such as the N2 antigen can be produced by a variety of techniques, including conventional monoclonal antibody methodology e.g., the standard somatic cell hybridization technique of Kohler and Milstein, Nature 256: 495 (1975). Although somatic cell hybridization procedures are preferred, in principle, other techniques for producing monoclonal antibody can be employed, e.g., viral or oncogenic transformation of B lymphocytes. An exemplary animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Human monoclonal antibodies can, for example, be generated using transgenic mice carrying the human immunoglobulin genes rather than mouse immunoglobulin genes. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immuno. 17:33-40; Tuaillon et al. 1993 PNAS 90:3720-3724; Bruggeman et al. 1991 Eur. J. Immunol. 21:1323-1326). In one embodiment, hybridomas can be generated from human CD5⁺, B-1 cells. Alternatively, “humanized” murine hybridomas can be used that recognize cross-reactive ischemic antigen.

Monoclonal antibodies can also be generated by other methods known to those skilled in the art of recombinant DNA technology. For example, an alternative method, referred to as the “combinatorial antibody display” method, has been developed to identify and isolate antibody fragments having a particular antigen specificity, and can be utilized to produce monoclonal antibodies (for descriptions of combinatorial antibody display see e.g., Sastry et al. 1989 PNAS 86:5,728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al., 1991, Biotechniques 11:152-156). A similar strategy can also been used to amplify human heavy and light chain variable regions from human antibodies (Larrick et al., 1991, Methods: Companion to Methods in Enzymology 2:106-110).

The antibody inhibitors also encompass the specific antibodies described above including one or more modifications. In certain embodiments, the V region domains of heavy and light chains can be expressed on the same polypeptide, joined by a flexible linker to form a single-chain Fv fragment, and the scFV gene subsequently cloned into the desired expression vector or phage genome. As generally described in McCafferty et al., Nature (1990) 348:552-554, complete V_{H} and V_{L} domains of an antibody, joined by a flexible (G1y₄-Ser)₃ (SEQ ID NO: 35) linker can be used to produce a single chain antibody which can render the display package separable based on antigen affinity. Isolated scFV antibodies immunoreactive with the antigen can subsequently be formulated into a pharmaceutical preparation for use in the subject method.

An antibody that can be used according to the present invention can be one in which the variable region, or a portion thereof, e.g., the complementarity determining regions (CDR or CDRs), are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention. Any modification is within the scope of the invention so long as the antibody has at least one antigen binding portion.

Chimeric antibodies (e.g., mouse-human monoclonal antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (See Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl. Cancer Inst. 80:1553-1559).

A chimeric antibody can be further humanized by replacing sequences of the Fv variable region which are not directly involved in antigen binding with equivalent sequences from human Fv variable regions. General methods for generating humanized antibodies are provided by Morrison, S. L., 1985, Science 229:1202-1207 by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPII_{b}IIIₐ antibody producing hybridoma. The recombinant DNA encoding the chimeric antibody can then be cloned into an appropriate expression vector. Suitable humanized antibodies can alternatively be produced by CDR substitution. U.S. Pat. No. 5,225,539; Jones et al. 1986 Nature 321:552-525; Verhoeyan et al. 1988 Science 239:1534; and Beidler et al. 1988 J Immunol. 141:4053-4060.

Humanized or CDR-grafted antibodies can be produced by CDR-grafting or CDR substitution, wherein one, two, or all CDRs of an immunoglobulin chain can be replaced. See, for example, U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

A humanized or CDR-grafted antibody will have at least one or two but generally all recipient CDRs (of heavy and/or light immunoglobulin chains) replaced with a donor CDR. Preferably, the donor will be a rodent antibody, e.g., a rat or mouse antibody, and the recipient will be a human framework or a human consensus framework. In one embodiment, the donor is a mouse antibody, for example, the 21G6 and/or 12A6 antibodies as described above. Typically, the immunoglobulin providing the CDRs is called the “donor” and the immunoglobulin providing the framework is called the “acceptor.” In one embodiment, the donor immunoglobulin is a non-human (e.g., rodent). The acceptor framework can be a naturally-occurring (e.g., a human) framework or a consensus framework, or a sequence about 85% or higher, preferably 90%, 95%, 99% or higher identical thereto. All of the CDRs of a particular antibody may be replaced with at least a portion of a non-human CDR or only some of the CDRs may be replaced with non-human CDRs. It is only necessary to replace the number of CDRs required for binding of the humanized antibody to the Fc receptor.

Also within the scope of the invention are chimeric and humanized antibodies in which specific amino acids have been substituted, deleted or added. In some examples, humanized antibodies have amino acid substitutions in the framework region, such as to improve binding to the antigen. For example, a humanized antibody will have framework residues identical to the donor framework residue or to another amino acid other than the recipient framework residue. As another example, in a humanized antibody having mouse CDRs, amino acids located in the human framework region can be replaced with the amino acids located at the corresponding positions in the mouse antibody. Another example of a humanized antibody is a murine monoclonal antibody having a murine variable region but modified to have a human Fc region. Such substitutions are known to improve binding of humanized antibodies to the antigen in some instances.

Additionally, amino acid substitutions, deletions or additions may be made to the antibodies described herein to inhibit or block inflammation. For example, asparagine at position 297 of the IgG constant region may be substituted by alanine (N297A) to reduce glycosylation and thereby ability to activate complement and bind Fc receptor. (See e.g., Leatherbarrow R J, et al. (1985) Effector functions of a monoclonal aglycosylated mouse IgG2a: binding and activation of complement component C1 and interaction with human monocyte Fc receptor. Mol Immunol 22(4):407-415; Tao M H & Morrison S L (1989) Studies of aglycosylated chimeric mouse-human IgG. Role of carbohydrate in the structure and effector functions mediated by the human IgG constant region. (Translated from eng) J Immunol 143(8):2595-2601; and Kabat (1987) Sequences of Proteins of Immunological Interest (In: US Department of Human Services). The contents of each of these references are expressly incorporated herein by reference.

Antibody fragments of the invention can be obtained using conventional procedures known to one of skill in the art. For example, digestion of an antibody with pepsin yields F(ab′)2 fragments and multiple small fragments. Mercaptoethanol reduction of an antibody yields individual heavy and light chains. Digestion of an antibody with papain yields individual Fab fragments and the Fc fragment.

As will be understood, the IgM inhibitor, for example the peptide or antibody inhibitor can be administered in a pharmaceutical composition. A “therapeutically effective amount” or an “effective amount” is an amount which, alone or in combination with one or more other active agents, can control, decrease, inhibit, ameliorate, prevent or otherwise affect and/or achieve a recited effect (for example, protection or retention of cardiac function). An effective amount of the agent to be administered can be determined using methods well-known in the art. One of skill in the art would take into account the mode of administration, the disease or condition (if any) being treated and the characteristics of the subject, such as general health, other diseases, age, sex, genotype, body weight and tolerance to drugs. A “patient” can refer to a human subject in need of treatment.

As will be understood, the form of an agent or pharmaceutical composition depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

For parenteral administration, pharmaceutical compositions or pharmacologic agents can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

The compositions can be prepared as injectable formulations, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions and pharmacologic agents described herein can be administered in the form of a depot injection or implant preparation which can be formulated in such a manner as to permit a sustained or pulsatile release of the active ingredient.

The invention is illustrated by the following non-limiting example.

### EXEMPLIFICATION

### Example 1

### N2 and 21G6 Monoclonal Antibody (mAb) Administration Prior to Reperfusion Protected Cardiac Function in a Porcine Model

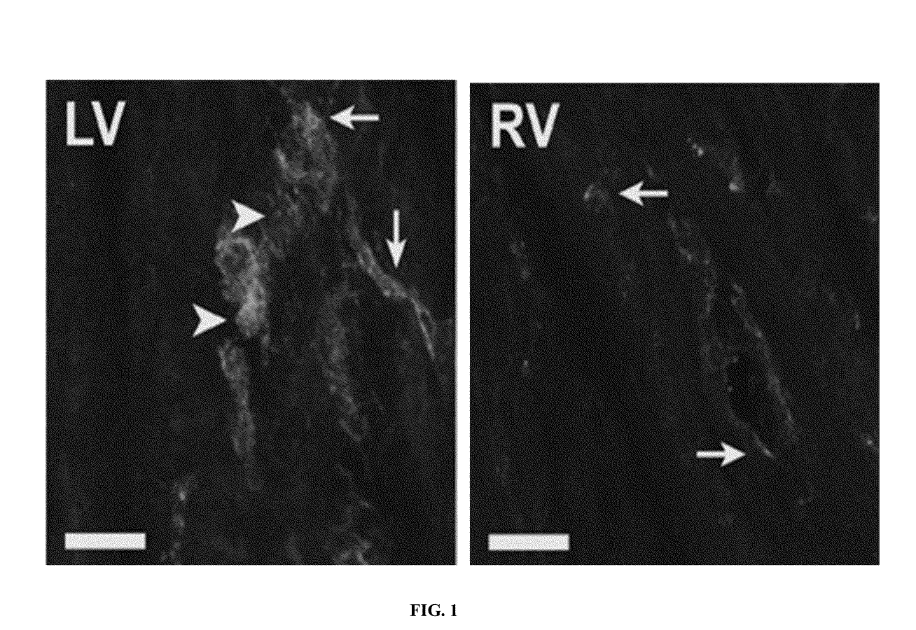

The effect of N2 peptide and the 21G6 (murine) antibody treatment on cardiac function in a porcine model of myocardial infarction was studied as described below. The porcine model has been described, for example, in McCall et al. (2012). Myocardial infarction and intramyocardial injection models in swine. Nat. Protoc. 7(8): 1479-1496, the contents of which are expressly incorporated by reference herein. As described above, the 21G6 mAb has been described in U.S. Patent Application Publication No. 20120093835A1, the contents of which are expressly incorporated by reference herein. Pilot studies (FIG. 1) indicated that fluorescently-labeled murine 21G6 mAb administered in vivo bound the N2 neo-epitope in vessels in the left ventricle (LV) but not in the uninjured right ventricle (RV), as expected.

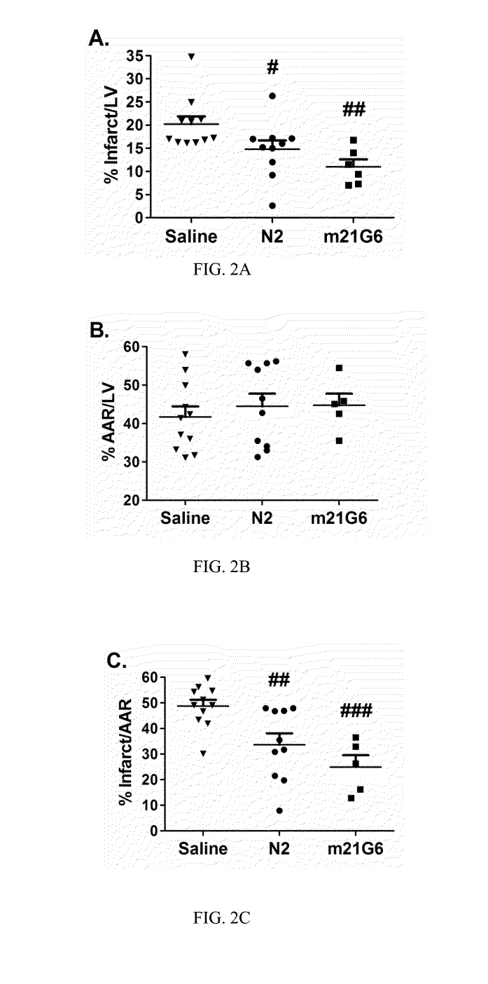

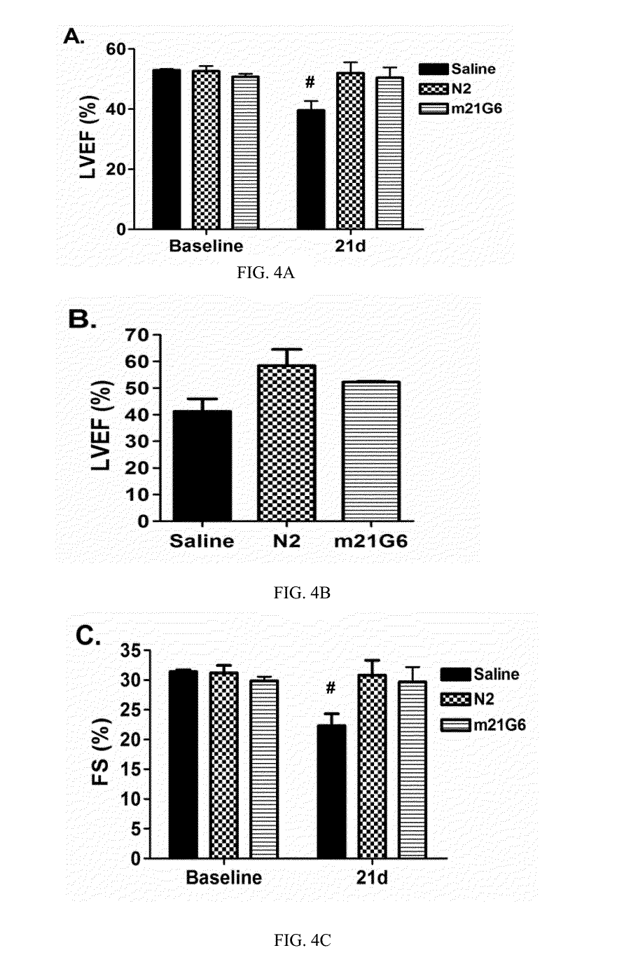

Intravenous (i.v.) administration of either N2 peptide (SEQ ID NO: 1) at 4.6 mg/kg 5 min prior to reperfusion or murine 21G6 mAb (2 mg/kg) 30 min prior to reperfusion resulted in significant protection from myocardial necrosis at 5d post-reperfusion as determined by TTC staining Specifically, treatment of N2 peptide resulted in a 31% reduction of infarct size while murine 21G6 Mab reduced infarct size by 49% as a percentage of the area at risk (AAR) (FIG. 2A-C). As expected, there was no difference in size of the AAR across the treatment groups indicating consistency in the coronary occlusion procedure throughout the study (FIG. 4B).

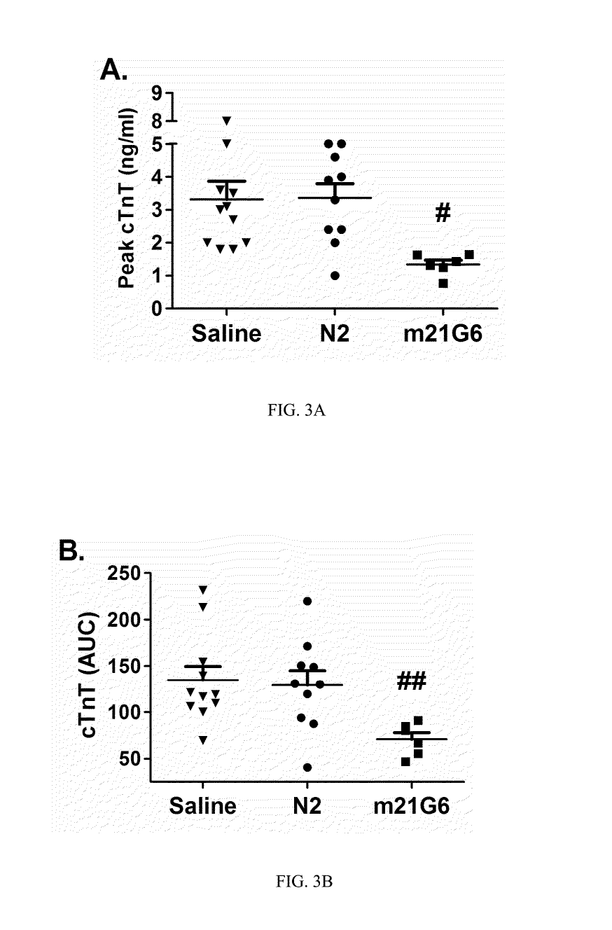

Cardiac troponin-T (cTnT), a well-established marker for cardiac tissue injury, from 8-120 h post-reperfusion, was also analyzed. While N2 peptide had no effect on either peak serum cTnT levels or area under the curve (AUC), cTnT levels as determined over the five day reperfusion period, murine 21G6 Mab resulted in a 60% reduction of peak serum cTnT levels and a 47% reduction of cTnT AUC values as compared to saline controls (FIG. 3A-B).

To determine the effect of N2 peptide and the 21G6 antibody treatment on cardiac function, echocardiography was employed. Echocardiography is widely accepted for evaluation of cardiac function and is utilized in emergency and operating rooms as well as intensive care departments in most hospitals. Ventricular function as assessed by echocardiography is accompanied by low intra-observer and inter-observer variability and assessment of left ventricular ejection fraction remains the standard of care in clinical practice. In addition, fractional shortening (FS), another measure of left ventricular function, was also calculated based on two-dimensional (2D) measurements at all time points measured at mid-ventricle and is defined as the difference between end-diastolic and end-systolic dimensions divided by end-diastolic dimension.

For animals in which functional data was generated (N2: n of 3, murine 21G6 mAb: n of 3, Saline: n of 3), cardiac imaging was performed via transthoracic echocardiography (TTE) at baseline (prior to occlusion procedure), and then up to 21d following reperfusion. For baseline TTE images were obtained using a high-frequency 2D probe and a Philips iE33 xMATRIX scanner (Andover, Mass.). Prior to sacrifice at 21 days, both 2D and 3D epicardial echocardiography images were obtained after median sternotomy was performed. 3D volumetric data sets were ECG-gated from 4-7 consecutive heartbeats. Additionally, 3D left ventricular volumes and ejection fractions were computed from the epicardial 3D datasets.

Prior to the occlusion procedure, a baseline TTE image was recorded. Afterwards, TTE was performed. Animals were treated in an identical manner as in the 5 day study for the catheterization procedure except that animals were maintained for an additional 16 days. The echocardiographer was blinded to the treatment schedule.

A single bolus administration of N2 peptide at the point of reperfusion dramatically improved cardiac function as measured by LVEF with 2D and 3D TTE (FIG. 4). At baseline, saline, N2- and murine 21G6 mAb -treated animals had LVEFs of 53%, 52.6%, and 51%, respectively. Following 21 d post-reperfusion, saline, N2-, and murine 21G6 mAb-treated animals had ejection fractions (EFs) of 39.6%, 52.0%, and 50.4%, respectively as calculated by 2D TTE using Quinones method (described in Quinones, et al. A new, simplified and accurate method for determining ejection fraction with two-dimensional echocardiography. Circulation 64:744, 1981, the contents of which are expressly incorporated by reference herein). Therefore, both N2- and murine 21G6 Mab-treated animals recovered 98.8% of their cardiac function as compared to 74.7% in the saline-treated animals. Only the saline-treated group at 21 d was significantly different from baseline.

Because geometric assumptions of an ellipsoid shape may not be accurate in a post-infarct model, the ejection fraction was also evaluated with 3D TTE. At 21 days post-reperfusion, N2- and murine 21G6 Mab-treated animals resulted in a 29% and 21% increase in EF, respectively, when compared to the saline controls at the same time point. These striking differences (p=0.089 for N2 and p=0.082 for murine 21G6 Mab) are particularly noteworthy in the context of our current sample size (n of 3 per treatment group). Additionally, FS, another measure of cardiac function, was also assessed throughout the 21d reperfusion period. Remarkably, as compared to the baseline controls, N2- and murine 21G6 mAb-treated animals recovered 98.8% and 99.4% of their cardiac function.

### Methods

Intravenous amiodarone infusion (1 mg/min)
Systemic heparinization (˜150 units/kg)
Percutaneously access of LAD via right (R) femoral artery
Occlusion mid-LAD with balloon catheter for 60 min as follows: Using a fluoroscopically guided balloon catheter approach, the left anterior descending coronary artery (LAD) was occluded for 60 min at a defined point distal to the 2^{nd} and 3^{rd} diagonal branches. Saline-treated animals developed approximately a 20% infarct of the left ventricle as assessed by TTC staining at 5d post-reperfusion as predicted
Serial EKGs to document ST elevation
Angiogram q20 min to confirm complete occlusion
Lidocaine and defibrillation as needed
Administration of control vs. N2 peptide 5 minutes prior to reperfusion or mAb 21G6 30 minutes before reperfusion
Measure trend in cardiac enzymes (CK, cTnT)
Cardiectomy on Day 5 or Day 21 via cardioplegic arrest
LAD ligation at site of occlusion followed by injection of Evans blue dye via aortic cannula gives estimate of Area At Risk (AAR)
Incubation of transverse sections in TTC for 15 min at 37° C.
Calculate % infarction by computerized planimetry
21G6 Alexa 568(1 mg/ml) infusion was initiated 2 min prior to reperfusion at ≈1 m1/min for ≈30 min via coronary catheter (results shown in FIG. 1 showing localization of the antibody to the injured left ventricle and not to the uninjured right ventricle)
Heart was perfused with cardioplegic solution 1 h post-ischemia, harvested, sectioned, and punch biopsies taken in the area at risk and in right ventricle
Sections were stained with anti-swine CD31 FITC (Serotec) 1/20 dilution
Using a fluoroscopically guided balloon catheter approach, the left anterior descending coronary artery (LAD) was occluded for 60 min at a defined point distal to the 2^{nd} and 3^{rd} diagonal branches. Saline-treated animals developed approximately a 20% infarct of the left ventricle as assessed by TTC staining at 5d post-reperfusion as predicted.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of protecting cardiac function in a subject in need thereof comprising administering to said subject an effective amount of an inhibitor of the interaction between a pathogenic IgM and the N2 epitope in cardiac tissue.

2. The method of claim 1, wherein the subject is a human subject.

3. The method of claim 1, wherein the protection of cardiac function comprises a protection from loss of left ventricular ejection fraction (LVEF).

4. The method of claim 1, wherein the protection comprises a protection from loss of fractional shortening.

5. The method of claim 2, wherein the subject has suffered myocardial infarction and the inhibitor is administered after the subject has suffered myocardial infarction.

6. The method of claim 2, wherein the subject is suffering from or is at risk of suffering an ischemic cardiac event.

7. (canceled)

8. The method of claim 1, wherein the subject is at risk for reperfusion injury and wherein administration of the inhibitor results in a protection from loss of LVEF.

9. The method of claim 8, wherein the subject has suffered myocardial infarction.

10. The method of claim 8, wherein the inhibitor is administered before reperfusion.

11. The method of claim 8, wherein the inhibitor is administered during reperfusion.

12. The method of claim 1, wherein the inhibitor is administered before and/or during a surgical procedure.

13. The method of claim 1, wherein the inhibitor is isolated antibody, or an antigenic fragment thereof.

14-19. (canceled)

20. The method of claim 13, wherein the antibody or fragment thereof possesses the epitopic specificity of the 12A6 antibody or the 21G6 antibody.

21. The method claim 13, wherein the antibody or fragment thereof has heavy chain CDR1, CDR2, and CDR3 that have the same amino acid sequence of the heavy chain CDR1, CDR2 and CDR3 of the 21G6 antibody and wherein the antibody or fragment thereof has the light chain CDR1, CDR2, and CDR3 that have the same amino acid sequence of the light chain CDR1, CDR2 and CDR3 of the 21G6 antibody.

22. The method of claim 20, wherein the antibody is human or humanized antibody or a fragment thereof.

23. The method of claim 8, wherein the LVEF is protected such that the LVEF after reperfusion is about 15% or less than that before reperfusion.

24. The method of claim 23, wherein the LVEF is protected such that the LVEF after reperfusion is about 10% or less than that before reperfusion.

25. The method of claim 23, wherein the LVEF is protected such that the LVEF after reperfusion is about 5% or less than that before reperfusion.

26. A method of treating a human subject at risk for reduced cardiac function after reperfusion comprising administering to said patient an effective amount of an inhibitor of the interaction between a pathogenic IgM and the N2 epitope in cardiac tissue prior to and/or during reperfusion therapy.

27. (canceled)

28. The method of claim 26, wherein the subject is at risk for reduced LVEF.

29. The method of claim 26, wherein the inhibitor is administered prior to reperfusion.

30. The method of claim 26, wherein the inhibitor is isolated antibody, or an antigenic fragment thereof.

31-36. (canceled)

37. The method of claim 30, wherein the antibody or fragment thereof possesses the epitopic specificity of the 12A6 antibody or the 21G6 antibody.

38. The method claim 30, wherein the antibody or fragment thereof has heavy chain CDR1, CDR2, and CDR3 that have the same amino acid sequence of the heavy chain CDR1, CDR2 and CDR3 of the 21G6 antibody and wherein the antibody or fragment has light chain CDR1, CDR2, and CDR3 that have the same amino acid sequence of the light chain CDR1, CDR2 and CDR3 of the 21G6 antibody.

39. The method of claim 38, wherein the antibody is human or humanized antibody or a fragment thereof.

40. A method of protecting LVEF in a patient that has suffered myocardial infarction comprising administering to said patient an effective amount of an inhibitor of the interaction between a pathogenic IgM and the N2 epitope in cardiac tissue prior to and/or during reperfusion therapy.

41. The method of claim 40, wherein the inhibitor is administered prior to reperfusion.

42. The method of claim 40, wherein the inhibitor is isolated antibody, or an antigenic fragment thereof.

43-48. (canceled)

49. The method of claim 41, wherein the antibody or fragment thereof possesses the epitopic specificity of the 12A6 antibody or the 21G6 antibody.

50. The method claim 41, wherein the antibody or fragment thereof has heavy chain CDR1, CDR2, and CDR3 that have the same amino acid sequence of the heavy chain CDR1, CDR2 and CDR3 of the 21G6 antibody and thereof has the light chain CDR1, CDR2, and CDR3 that have the same amino acid sequence of the light chain CDR1, CDR2 and CDR3 of the 21G6 antibody.

51. The method of claim 49, wherein the antibody is human or humanized antibody or a fragment thereof.
